(19) United States
(12) Patent Application Publication
(10) Pub. No.: US 2016/0287678 A1
(43) Pub. Date: Oct. 6, 2016

(54) GENE DELIVERY METHODS AND COMPOSITIONS

(71) Applicant: Agenovir Corporation, South San Francisco, CA (US)

(72) Inventor: Jianbin Wang, South San Francisco, CA (US)

(21) Appl. No.: 15/088,482

(22) Filed: Apr. 1, 2016

Related U.S. Application Data
(60) Provisional application No. 62/142,192, filed on Apr. 2, 2015.

(51) Int. Cl. A61K 38/46; A61M 25/10; A61M 5/00; A61K 9/00; C12N 15/113

(52) U.S. Cl. CPC A61K 38/465; A61K 9/0019; C12N 15/1131; A61M 5/007; A61M 25/10; C12Y 301/00; C12N 2320/32; C12N 2310/3513; A61M 2205/52; A61M 2025/105

(57) ABSTRACT

The invention provides methods and compositions that remove target genetic material from a subject by hydrodynamic delivery of an enzyme that degrades the target genetic material. The methods include delivering a solution include a nucleic acid, protein, or ribonucleoprotein to a blood vessel at a pressure sufficient to cause the material to enter cells proximal to the blood vessel. The enzyme may be Cas9 and may be provided with a guide RNA. The target genetic material may be viral genome with the guide RNA complementary to a portion of the viral genome.

### CROSS-REFERENCE TO RELATED APPLICATION

This application claims the benefit of and priority to U.S. Provisional Application 62/142,192, filed Apr. 2, 2015, the contents of which are incorporated by reference.

### FIELD OF THE INVENTION

The invention generally relates to method of gene delivery.

### BACKGROUND

Some viral infections lie dormant in a subject for a long time in what is called viral latency. Latency is a period in the viral life cycle in which, after initial infection, viral proliferation ceases. However, the viral genome persists. In this relatively quiescent state, the virus can reactivate, causing acute infection and producing large amounts of progeny. While this can produce symptoms such as cold sores or warts, more serious ramifications of a latent infection include the possibility of transforming a cell, leading to uncontrolled cell division. For example, it is thought that latent infections of the human papilloma virus may lead to cervical cancer as a result of cellular transformation. The human immunodeficiency virus (HIV) is believed to persist as replication-competent HIV in resting CD4-positive T cells, despite prolonged exposure to antiretroviral drugs, which may explain the inability of antiretroviral treatment to cure HIV infection.

The herpes virus family (herpesviridae), HIV, hepatitis B and C viruses, and human papilloma viruses (HPV, BK, JC, MCV) all establish latent infection characterized by persistence of viral DNA. The herpes virus family includes varicella-zoster virus, Epstein-Barr virus, cytomegalovirus, and herpes simplex viruses (HSV-1, HSV-2). There are currently no available antiviral drugs capable of eliminating these persistent viruses. Hepatitis B is caused by the infection of the hepatitis B virus (HBV) and is the most common serious liver infection in the world. While the FDA has approved seven drugs to treat Hepatitis B, none of them provides a complete cure and provide no way to eradicate the latent virus. As a result, about 1 out of 5 people with chronic HBV infection die from the infection.

### SUMMARY

The invention provides methods and compositions that remove target genetic material such as foreign viral DNA from a subject by hydrodynamic delivery of an enzyme or nucleic acid that codes an enzyme that degrades the target genetic material. Hydrodynamic delivery involves delivering this enzyme in the form of one or a combination of nucleic acid, protein, and ribonucleoprotein (RNP) formulated in solution into a subject's vasculature under hydrodynamic pressure that helps permeabilize the vascular endothelium and the plasma membrane of surrounding cells. The nucleic acid, protein, or RNP is thus delivered into those cells. Where the nucleic acid is vector plasmid DNA, message RNA (mRNA), or guide RNA (gRNA) that encodes an enzyme or is part of the enzyme that will act against the target genetic material, expression of that enzyme allows it to degrade or otherwise interfere with the target genetic material. Where the enzyme may be a nuclease such as the Cas9 endonuclease protein combined with gRNA to form RNP that target the nuclease to the target genetic material. Where the target genetic material includes the genome of a virus, gRNA complementary to parts of that genome can guide the degredation of that genome by the nuclease, thereby preventing any further replication, viral gene expression, or even by removing any intact viral genome from the cells entirely. By these means, latent viral infections can be targeted for eradication. Since hydrodynamic gene delivery of nuclease with activity specific to the genome of a latent virus is provided, methods of the invention may be used to address latent viral infections. Thus methods and compositions of the invention may provide relief from the adverse consequences of viruses such as HBV, Epstein-Barr, or others.

In certain aspects, the invention provides a system for delivering a nuclease. The system includes a solution comprising a targetable nuclease, the targetable nuclease being an enzyme that cuts target genetic material in a sequence-specific manner; a delivery catheter dimensioned for insertion into a blood vessel of a subject; and a delivery mechanism operable to deliver the solution through the catheter at a pressure sufficient to cause the targetable nuclease to enter cells proximal to the blood vessel. The system is operated under principles of hydrodynamic delivery in the pressures can be brought to the point that helps permeabilize the vascular endothelium and the plasma membrane of surrounding cells or to just below that point. The target genetic material may be a portion of a viral genome. In some embodiments, the delivery catheter comprises a balloon, a delivery lumen for delivering the solution, and an inflation lumen for inflating the balloon. The targetable nuclease may be a transcription-activator like effector nuclease (TALEN), a zinc finger nuclease (ZFN), a meganuclease, or a CRISPR-associated nuclease. In some embodiments, the targetable nuclease is a CRISPR-associated nuclease and is delivered as part of a ribonucleoprotein (RNP) that includes a recombinant Cas9 protein combined with a guide RNA (gRNA). In some embodiments, the targetable nuclease is in a nanoparticle within the solution. The viral genome may be of a virus selected from the group consisting of a human papilloma virus (HPV), an Epstein-Barr virus (EBV), Kaposi's sarcoma-associated herpesvirus (KSHV), hepatitis B virus (HBV), hepatitis C virus (HCV), human T-cell lymphotrophic virus type I (HTLV-I), and Merkel cell polyomavirus (MCV). In preferred embodiments, the CRISPR-associated nuclease is Cas9, the guide RNA includes a 20 nucleotide portion at least 60% complementary to a target within the viral genome, and wherein the 20 nucleotide portion is not complementary to any location in a human genome.

The system may include a pressure sensor operable to measure pressure in the blood vessel. The delivery mechanism may use a computer with a processor coupled to non-transitory memory to control injection of the solution according to the measured pressure. Preferably, the delivery mechanism is operable to deliver the solution at an injection volume of less than 0.1 L/1 kg body weight of the subject. Most preferably, the delivery mechanism controls delivery to an injection volume between 0.5 and 5% of BW (i.e., between 5 and 50 mL/kg body weight). The system may include a phase contrast medium that can be injected into the blood vessel and a radiographic imaging subsystem for imaging the blood vessel with the contrast medium therein.

In certain embodiments, the system is operable to control injection to deliver the solution when the cells proximal to the blood vessel are tumor cells and the system is operable to cause the solution to be delivered into the tumor cells.

Aspects of the invention provide a method of cleaving nucleic acid in a subject. The method includes delivering a solution that includes a targetable nuclease to a blood vessel of the subject at a pressure sufficient to cause the targetable nuclease to enter cells proximal to the blood vessel. The targetable nuclease comprises an enzyme that cuts target genetic material in a sequence-specific manner (e.g., TALEN, ZFN, meganuclease, CRISPR-associated nuclease). The target genetic material may include a portion of a viral genome. The solution may be delivered via an intravascular delivery catheter. Preferably, the solution is delivered at an injection volume of less than 0.1 L/1 kg body weight of the subject. In some embodiments delivery comprises navigating the catheter through a femoral vein of the subject and into a hepatic vein or through a jugular vein of the subject and into a hepatic artery.

In some embodiments, the catheter is a balloon catheter. Delivery includes navigating the balloon catheter to the blood vessel at a target location in the subject, inflating the balloon, and delivering the solution via a lumen in the balloon catheter. The method may include imaging the blood vessel by injecting a phase contrast medium and using a radiographic imaging system; measuring pressure within the blood vessel via a sensor inserted therein; and using a computer comprising a processor coupled to non-transitory memory to control an injection speed of the solution according to the measured pressure.

In certain embodiments, the targetable nuclease is a CRISPR-associated nuclease delivered as part of a ribonucleoprotein that includes a guide RNA. The viral genome is of a virus selected from the group consisting of a human papilloma virus (HPV), an Epstein-Barr virus (EBV), Kaposi's sarcoma-associated herpesvirus (KSHV), hepatitis B virus (HBV), hepatitis C virus (HCV), human T-cell lymphotrophic virus type I (HTLV-I), and Merkel cell polyomavirus (MCV).

In certain embodiments, the method is used to treat a tumor and the cells proximal to the blood vessel are tumor cells.

In certain aspects, the invention provides methods for the removal of target genetic material. The methods include delivering a solution of a nucleic acid to a blood vessel of a subject at a pressure sufficient to cause the nucleic acid to enter cells proximal to the blood vessel, wherein the nucleic acid comprises a gene for an enzyme that cuts target genetic material.

The enzyme may be Cas9 and the solution may be delivered at a pressure sufficient to generate pores in the cells proximal to the blood vessel. The nucleic acid may be a plasmid comprising a cas9 gene and at least one gene for a guide RNA (gRNA) and the target genetic material may be viral genome, i.e., with the sgRNA complementary to a portion of the viral genome. In some embodiments, the viral genome is a hepatitis B genome and the plasmid contains genes for one or more gRNAs targeting locations in the hepatitis B genome such as PreS1, DR1, DR2, a reverse transcriptase (RT) domain of polymerase, an Hbx, the core ORF, or combinations thereof.

For hydrodynamic gene delivery, the solution may be delivered by injection or preferably via an intravascular delivery catheter. The delivery catheter may be navigated through a femoral vein of the subject and into a hepatic vein or through a jugular vein of the subject and into a hepatic artery. In certain embodiments, the catheter comprises a balloon catheter. The balloon catheter is navigated to the blood vessel at a target location in the subject, the balloon is inflated, and the solution is delivered via a lumen in the balloon catheter. The method may further include delivering a radiographic contrast agent and imaging the balloon catheter in the target location.

In certain embodiments, the target genetic material is genome of a virus and the nucleic acid is a plasmid comprising a cas9 gene and at least one gRNA targeting the genome of the virus. The plasmid may include a viral origin of replication (i.e., such that prospective replication of the latent virus leads to replication of the very plasmid genes targeting that virus). In an exemplary embodiment, the virus is hepatitis B and the sgRNA includes one or more of sgHBV-RT, sgHBV-Hbx, sgHBV-Core, and sg-HBV-PerS1.

Aspects of the invention provide a system for delivering a nuclease. The system includes a solution comprising a nucleic acid encoding a targetable nuclease (which includes an enzyme that cuts target genetic material in a sequence-specific manner), a delivery catheter dimensioned for insertion into a blood vessel of a subject, and a delivery mechanism operable to deliver the solution through the catheter at a pressure sufficient to cause the nucleic acid to enter cells proximal to the blood vessel. Preferably, the target genetic material comprises a portion of a viral genome. The targetable nuclease may include a TALEN, a ZFN, a meganuclease, or a CRISPR-associated nuclease. Preferably, the delivery catheter comprises a balloon, a delivery lumen for delivering the solution, and an inflation lumen for inflating the balloon.

In certain embodiments, the targetable nuclease is a CRISPR-associated nuclease and the nucleic acid also encodes a guide RNA. In preferred embodiments, the CRISPR-associated nuclease is Cas9, the guide RNA includes a 20 nucleotide portion at least 60% complementary to a target within the viral genome, and wherein the 20 nucleotide portion is not complementary to any location in a human genome.

The nucleic acid may be provided in any suitable form including, for example, a plasmid, a viral vector such as an AAV, or an mRNA. The viral genome may be of a virus selected from the group consisting of a human papilloma virus (HPV), an Epstein-Barr virus (EBV), Kaposi's sarcoma-associated herpesvirus (KSHV), hepatitis B virus (HBV), hepatitis C virus (HCV), human T-cell lymphotrophic virus type I (HTLV-I), and Merkel cell polyomavirus (MCV).

Preferably, the system includes a pressure sensor operable to measure pressure in the blood vessel. The delivery mechanism may include a computer comprising a processor coupled to non-transitory memory to control injection of the solution according to the measured pressure. The system may include a phase contrast medium that can be injected into the blood vessel and a radiographic imaging subsystem for imaging the blood vessel with the contrast medium therein. Preferably, the delivery mechanism is operable to deliver the solution at an injection volume of less than 0.1 L/1 kg body weight of the subject.

The system may be operable to control injection to deliver the solution when the cells proximal to the blood vessel are tumor cells and the system is operable to cause the solution to be delivered into the tumor cells.

### BRIEF DESCRIPTION OF THE DRAWINGS

FIG. 1 diagrams a method for cleaving target genetic material within a subject.

FIG. 2 shows portions of a viral genome targeted by a targetable nuclease.

FIG. 3 shows a gel resulting from an in vitro cleavage of target genetic material.

FIG. 4 shows a nucleic acid according to certain embodiments.

FIG. 5 shows a catheter useful for hydrodynamic delivery.

FIG. 6 shows a system for delivering a targetable nuclease.

FIG. 7 shows components of a delivery mechanism.

FIG. 8 shows a targetable nuclease.

FIG. 9 shows a catheter for delivery of a targetable nuclease or a nucleic acid.

FIG. 10 diagrams a method of cleaving nucleic acid in a subject.

### DETAILED DESCRIPTION

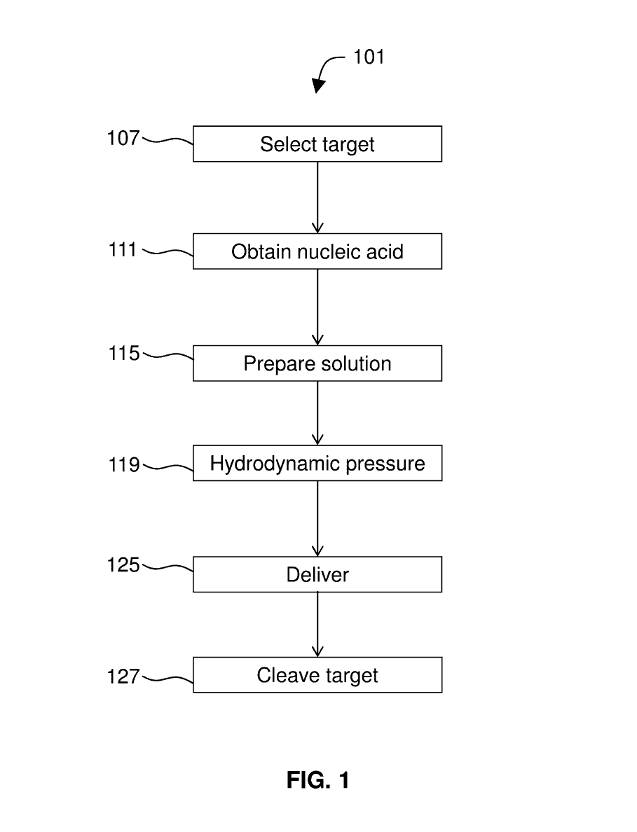

FIG. 1 diagrams a method 101 for removing target genetic material from a subject. The method 101 may include the steps of selecting 107 a target, and this may specifically include choosing guide RNAs when the targetable nucleic acid will be a CRISPR-associated nuclease. The method 101 may include obtaining 111 a nucleic acid, such as a plasmid, an mRNA, or a viral vector (e.g., AAV) that encodes the targetable nuclease. The method 101 may include the step of preparing 115 a solution that includes the nucleic acid. The method 101 may include using a delivery mechanism (e.g., a pump or a syringe) to generate 119 hydrodynamic pressure. The method 101 includes delivering 125 the solution comprising the nucleic acid to a blood vessel of a subject at a pressure sufficient to cause the nucleic acid to enter cells proximal to the blood vessel, wherein the nucleic acid comprises a gene for an enzyme that cuts target genetic material. The method 101 includes causing the targetable nucleic acid to cleave 127 the target genetic material.

Targeted endonuclease technologies, such as zinc finger nuclease (ZFN), transcription activator-like effector nuclease (TALEN), and clustered regularly interspaced short palindromic repeats (CRISPR), have provided great promise to gene therapy (Cell Stem Cell. 2013, 13(6): 653-8). By targeting viral DNA, recent studies demonstrated the treatment of latent viral infections in human cells with CRISPR. See Wang & Quake, 2014, RNA-guided endonuclease provides a therapeutic strategy to cure latent herpesviridae infection, PNAS 111(36):13157-13162 and Hu et al., 2014, RNA-directed gene editing specifically eradicates latent and prevents new HIV-1 infection, PNAS 111(31):11461-6, both incorporated by reference. Methods and materials of the present invention may be used to apply targeted endonuclease to specific genetic material such as a latent viral genome like HBV. The invention further provides for the efficient and safe delivery of nucleic acid (such as a DNA plasmid) into target cells (e.g., hepatocytes).

In an exemplary embodiment, the invention provides a combination of hydrodynamic gene delivery and targeted endonuclease to treat chronic HBV infection.

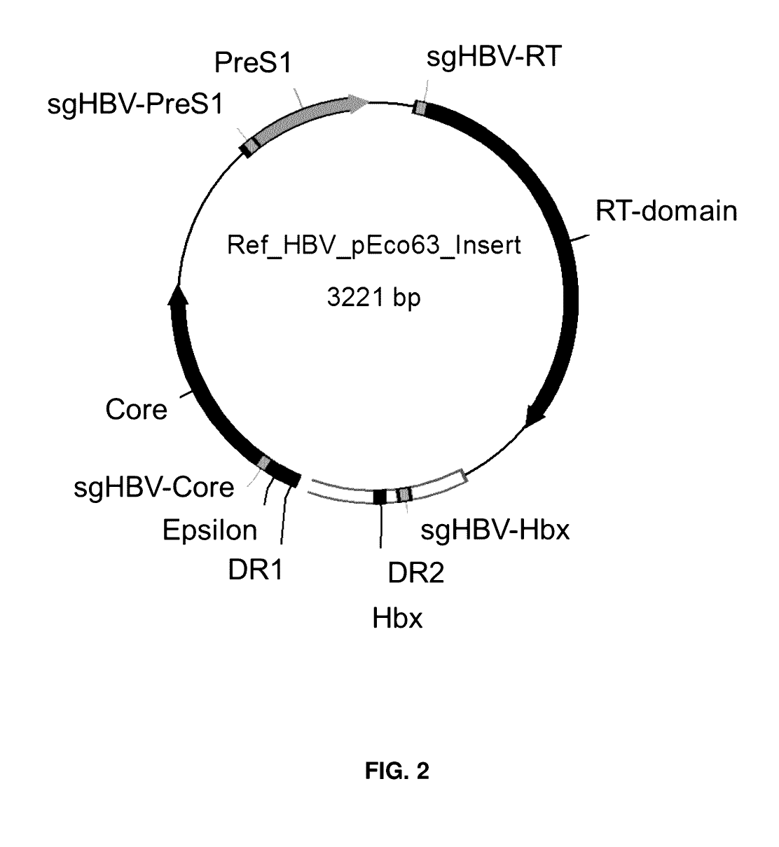

FIG. 2 diagrams the HBV genome. In some embodiments, the invention uses one or several guide RNAs against key features within a genome such as the HBV genome shown in FIG. 2. With reference to FIG. 2, HBV starts its infection cycle by binding to the host cells with PreS1. Guide RNA against PreS1 locates at the 5′ end of the coding sequence. Endonuclease digestion will introduce insertion/deletion, which leads to frame shift of PreS1 translation. HBV replicates its genome through the form of long RNA, with identical repeats DR1 and DR2 at both ends, and RNA encapsidation signal epsilon at the 5′ end. The reverse transcriptase domain (RT) of the polymerase gene converts the RNA into DNA. Hbx protein is a key regulator of viral replication, as well as host cell functions. Digestion guided by RNA against RT will introduce insertion/deletion, which leads to frame shift of RT translation. Guide RNAs sgHbx and sgCore can not only lead to frame shift in the coding of Hbx and HBV core protein, but also deletion the whole region containing DR2-DR1-Epsilon. The four gRNAs in combination can also lead to systemic destruction of HBV genome into small pieces.

FIG. 2 shows key parts in the HBV genome targeted by CRISPR guide RNAs.

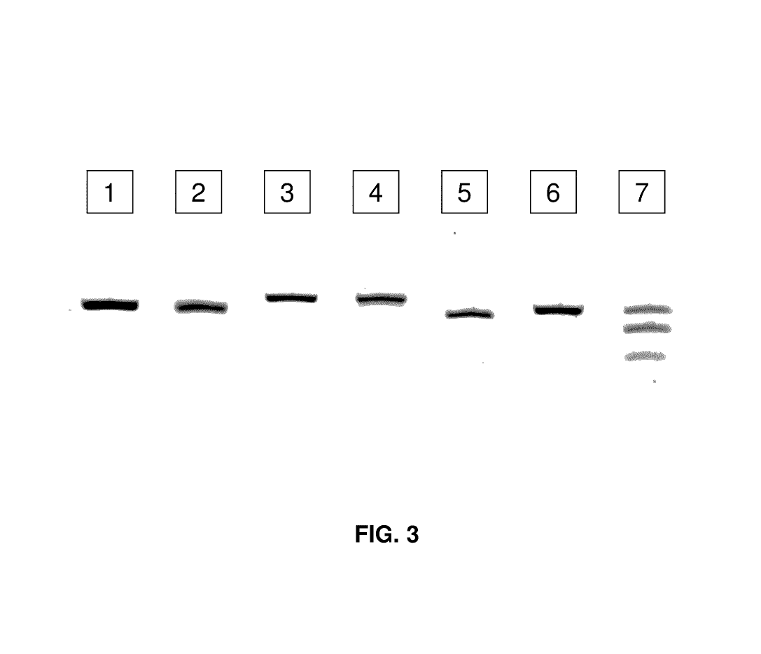

FIG. 3 shows a gel resulting from an in vitro CRISPR assay against HBV. Lane 1, 3, and 6 include PCR amplicons of HBV genome flanking RT, Hbx-Core, and PreS1, respectively. Lanes 2, 4, 5, and 7 include PCR amplicons treated with sgHBV-RT, sgHBV-Hbx, sgHBV-Core, sgHBV-PreS1, respectively. The presence of multiple fragments especially visible in lanes 5 and 7 show that sgHBV-Core and sgHBV-PreS1 provide especially attractive targets in the context of HBV and that use of systems and methods of the invention may be shown to be effective by an in vitro validation assay. The targetable nuclease forms a complex with the gRNA (e.g., crRNA+tracrRNA or sgRNA). The complex cuts the viral nucleic acid in a targeted fashion to incapacitate the viral genome. The Cas9 endonuclease causes a double strand break in the viral genome. By targeted several locations along the viral genome and causing not a single strand break, but a double strand break, the genome is effectively cut a several locations along the genome. In a preferred embodiment, the double strand breaks are designed so that small deletions are caused, or small fragments are removed from the genome so that even with repair mechanisms, the genome is render incapacitated.

The invention provides the aforementioned guide RNAs. To demonstrate, an in vitro assay was performed with cas9 protein and DNA amplicons flanking the target regions. As shown in FIG. 2, DNA electrophoresis shows strong digestion at the target sites.

To achieve the CRISPR activity in cells, expression plasmids coding cas9 and guide RNAs are delivered to cells of interest (e.g., cells carrying HBV DNA). To demonstrate in an in vitro assay, anti-HBV effect may be evaluated by monitoring cell proliferation, growth, and morphology as well as analyzing DNA integrity and HBV DNA load in the cells.

To deliver the Cas9 and sgRNAs, the invention provides for the use of hydrodynamic gene delivery. This technology controls hydrodynamic pressure in capillaries to enhance endothelial and parenchymal cell permeability (Hydrodynamic Gene Delivery: Its Principles and Applications, Molecular Therapy (2007) 15 12, 2063-2069). The first clinical test of hydrodynamic gene delivery in humans was reported at the 9th Annual Meeting of the American Society of Gene Therapy (Clinical Study with Hydrodynamic Gene Delivery into Hepatocytes in Humans). Hydrodynamic gene delivery avoids potential host immune response seen in AAV delivery (Prolonged susceptibility to antibody-mediated neutralization for adeno-associated vectors targeted to the liver.).

Hydrodynamic gene delivery can also be applied to liver transplant (Hydrodynamic plasmid DNA gene therapy model in liver transplantation). Injection volumes of 40-70% of the liver weight have been found to be effective in gene delivery. It may be preferable to use an injection volume of 10% of body weight or less for hydrodynamic delivery. Methods and systems herein may be used for delivering a solution at an injection volume of about 1 or 2% of body weight. Specifically, about 15 mL of solution per kg body weight may be delivered (i.e., 1.5% BW). Most preferably, injection volume is between 0.5 and 5% BW.

Combination of hydrodynamic delivery with targeted endonuclease can potentially eliminate viral genetic material (e.g., such as HBV) from liver transplant, and provide more qualified organs.

The delivery of targeted endonuclease (e.g., Cas9+sgRNA) may be combined with conventional antiviral drugs, such as Lamivudine and Telbivudine. In such way, the viral load may be greatly reduced before endonuclease treatment to improve treatment efficacy.

For hydrodynamic delivery, a solution is delivered at a pressure sufficient to generate pores in the cells proximal to the blood vessel, or at a pressure just beneath that threshold. Hydrodynamic delivery is used to deliver a targetable nuclease or a nucleic acid such as a plasmid that encodes the targetable nuclease. In a preferred embodiment, the targetable nuclease is Cas9.

Cas9 (CRISPR associated protein 9) is an RNA-guided DNA endonuclease enzyme. Cas9 was found as part of the Streptococcus pyrogenes immune system, where it memorizes and later cuts foreign DNA by unwinding it to seek regions complementary to a 20 basepair spacer region of the guide RNA, where it then cuts. Cas9 can be used to make site-directed double strand breaks in DNA, which can lead to gene inactivation or the introduction of heterologous genes through non-homologous end joining and homologous recombination. Other exemplary tools for gene editing include zinc finger nucleases and TALEN proteins.

Cas9 can cleave nearly any sequence complementary to the guide RNA. Native Cas9 uses a guide RNA composed of two disparate RNAs that associate to make the guide—the CRISPR RNA (crRNA), and the trans-activating RNA (tracrRNA). Additionally or alternatively, Cas9 targeting may be simplified through the engineering of a chimeric single guide RNA.

Studies suggest that Cas9 contain RNase H and HNH endonuclease homologous domains which are responsible for cleavages of two target DNA strands, respectively. The sequence similar to RNase H has a RuvC fold (one member of RNase H family) and the HNH region folds as T4 Endo VII (one member of HNH endonuclease family). Previous works on Cas9 have demonstrated that HNH domain is responsible for complementary sequence cleavage of target DNA and RuvC is responsible for the non-complementary sequence. Methods and materials of the invention use a plasmid that includes a cas9 gene and at least one gene for a guide RNA (gRNA). The gRNA is complementary to a portion of the viral genome. As used herein, guide RNA refers to a single guide RNA (e.g., crRNA and trans-activating RNA on a single molecule), crRNA, or crRNA+tracrRNA. Thus gRNA means a single guide RNA (sgRNA) or a duplex guide RNA that includes a CRISPR RNA (crRNA) and a trans-activating crRNA (tracrRNA).

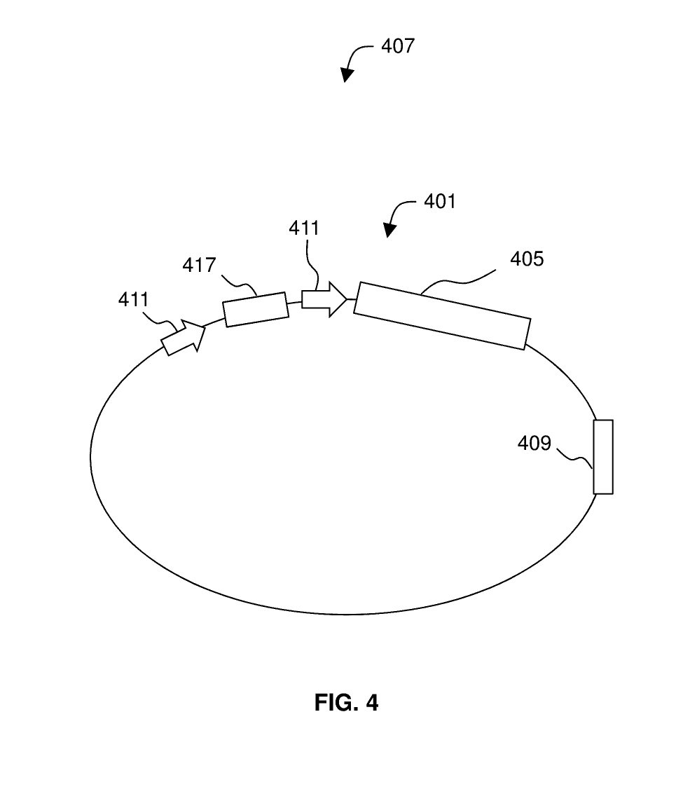

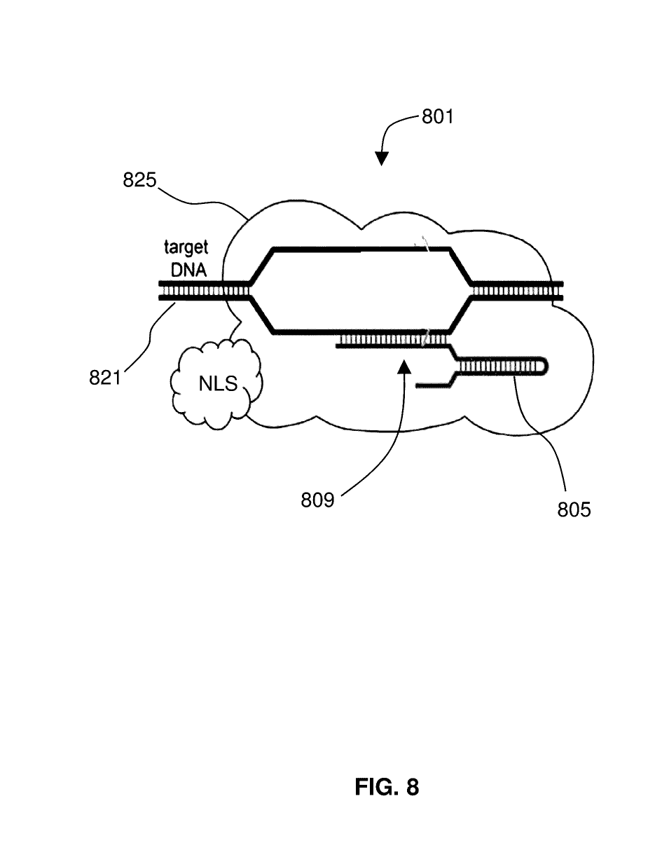

FIG. 4 shows a nucleic acid 401 encoding a targetable nuclease. The depicted nucleic acid is in the form of a plasmid, although in some embodiments, the nucleic acid 401 is in another form such as a viral vector like an adeno-associated viral (AAV) vector or an mRNA. The nucleic acid 401 includes a nuclease gene 405 that encodes a targetable nuclease. The targetable nuclease comprises an enzyme that cuts target genetic material in a sequence-specific manner. The nucleic acid 401 also optionally includes a guide RNA gene 417 that encodes a guide RNA. FIG. 8 shows a targetable nuclease 801. In FIG. 8, the targetable nuclease 801 is illustrated as a ribonucleoprotein (RNP) that includes Cas9 and a guide RNA 805. The nucleic acid 401 may optionally include an On gene 409, one or more promoter gene 411, other elements, or combinations thereof, as necessary or best-suited for methods and systems disclosed herein.

The nucleic acid 401 is provided within a solution 407 (e.g., saline) as part of a system for delivering a nuclease. The system includes the solution 407 that includes the nucleic acid 401 encoding the targetable nuclease 801. The system also includes a delivery catheter and a delivery mechanism (such as a pump or syringe) operable to deliver the solution 407 through the catheter at a pressure sufficient to cause the nucleic acid to enter cells proximal to the blood vessel.

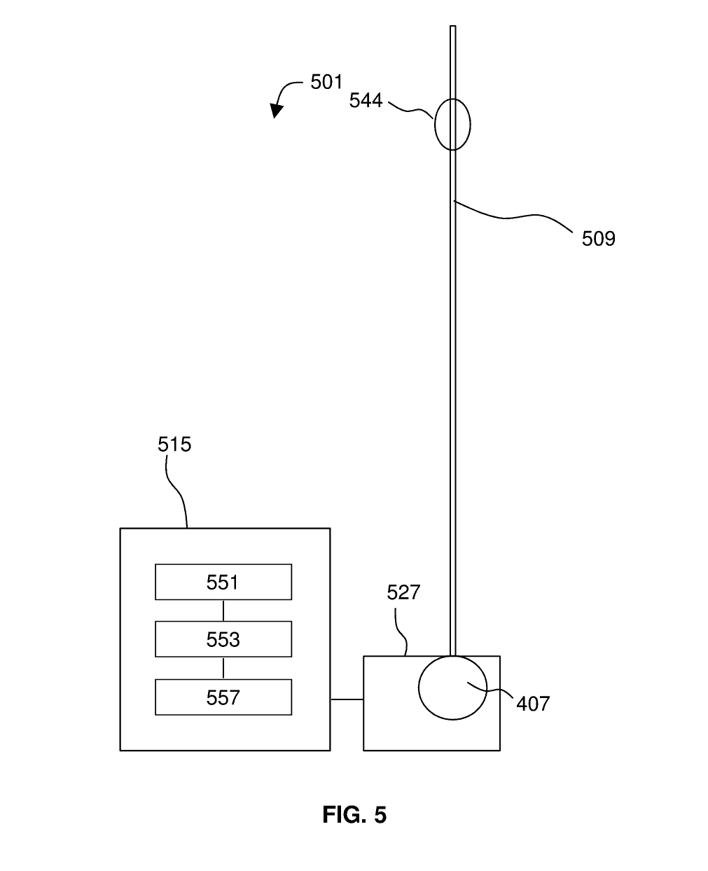

FIG. 5 shows a system 501 for delivering a nuclease. The system 501 includes the solution 407 that includes the nucleic acid 401 encoding the targetable nuclease 801. The targetable nuclease includes an enzyme that cuts target genetic material in a sequence-specific manner. The system 501 includes a delivery catheter 509 dimensioned for insertion into a blood vessel of a subject. In certain embodiments, the delivery catheter 509 is a 7 Fr catheter. The system 501 includes a delivery mechanism 527 operable to deliver the solution 407 through the catheter 509 at a pressure sufficient to cause the nucleic acid 401 to enter cells proximal to the blood vessel. The delivery mechanism 527 may be a pump and may be operably connected to, and under the control of, a computer 515. The computer 515 preferably includes a processor 553 coupled to a plurality of input/output device 551 and a memory subsystem 557. The input/output devices 551 may include a monitor, keyboard, mouse, internet card, modem, trackpad, touchpad, touchscreen, Ethernet port, Wi-Fi card, etc. The memory subsystem 557 may include RAM, ROM (e.g., a hard drive, solid state drive, flash memory, etc.), or both. The catheter 509 is useful for hydrodynamic delivery of the solution 407 containing the nucleic acid 401. The catheter preferably includes one or more of balloon 544. The nucleic acid 401 includes a nuclease gene 405 that encodes the targetable nuclease 801. The targetable nuclease 801 is an enzyme that cuts a target genetic material in a sequence-specific manner. The target genetic material is preferably a portion of a viral genome from a virus such as a human papilloma virus (HPV), an Epstein-Barr virus (EBV), Kaposi's sarcoma-associated herpesvirus (KSHV), hepatitis B virus (HBV), hepatitis C virus (HCV), human T-cell lymphotrophic virus type I (HTLV-I), and Merkel cell polyomavirus (MCV).

Where the viral genome is a hepatitis B genome, the nucleic acid 401 may contain genes 417 for one or more guide RNAs targeting locations in the hepatitis B genome such as PreS1, DR1, DR2, a reverse transcriptase (RT) domain of polymerase, an Hbx, and the core ORF. In a preferred embodiment, the one or more sgRNAs comprise one selected from the group consisting of sgHBV-Core and sgHBV-PreS1.

For hydrodynamic gene delivery, the solution 407 may be delivered via the intravascular delivery catheter 509, e.g., by navigating a balloon catheter to the blood vessel at a target location in the subject, inflating the balloon, and delivering the solution via a lumen in the balloon catheter.

While just described in terms of delivering a nucleic acid that encodes a targetable nuclease, it will be appreciated that methods and materials disclosed herein may be purposed for the delivery of the nuclease itself, e.g., in an active form.

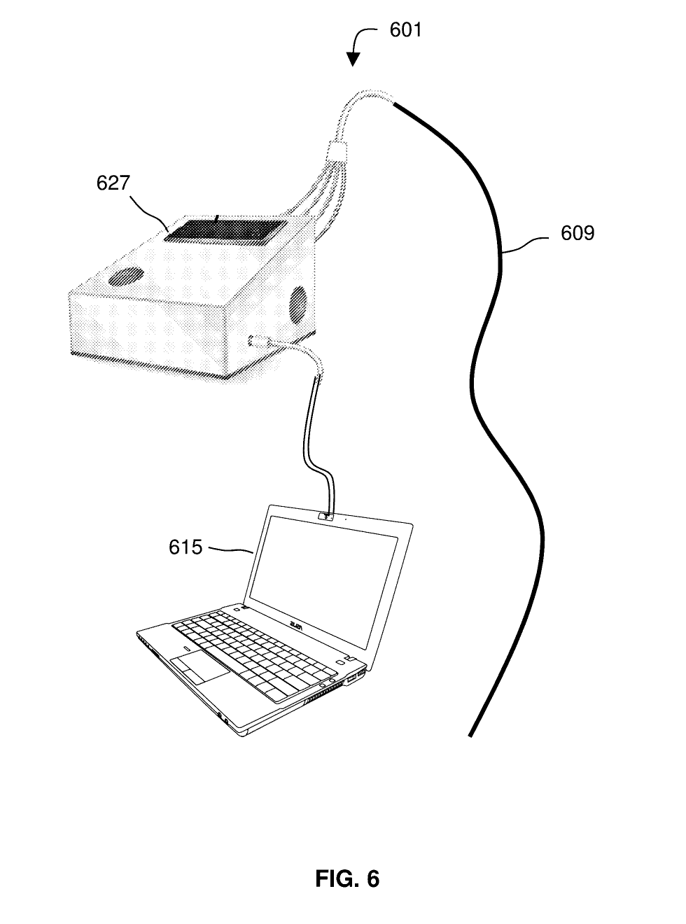

FIG. 6 shows a system 601 for delivering a nuclease. The components depicted in FIG. 6 may be substantially equivalent to those depicted in FIG. 5, except for the contents of the solution therein. The system 601 includes a delivery catheter 609 and a delivery mechanism 627. The delivery system 601 may include a computer 615 comprising a processor coupled to non-transitory memory to control injection of a solution. The solution may be provided from within the delivery mechanism 627.

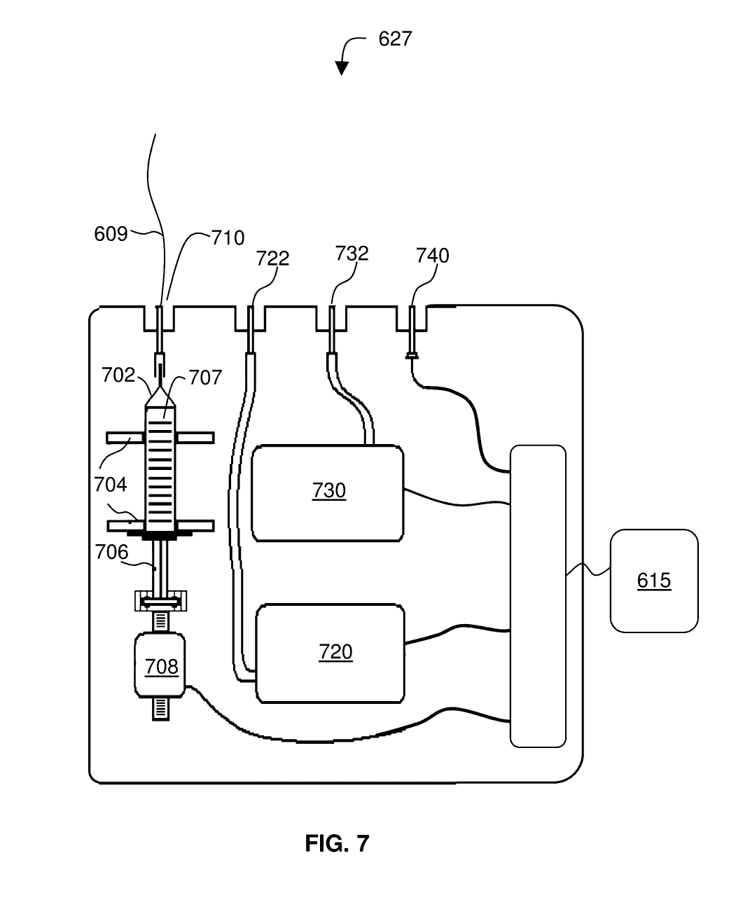

FIG. 7 shows components of the delivery mechanism 627 (or delivery mechanism 527). The delivery mechanism 627 includes a vessel 702. The vessel 702 is held in place by mounts 704 and includes a plunger 706 connected to an injection control unit 708. The control unit 508 may be a displacement-controlled stepper motor that regulates the speed of injection of a solution 707 contained in the vessel 702. The vessel 702 is coupled to an injection lumen of the catheter 609 by way of an injection coupling 710. The actuator 708 may be controlled by the computer 615.

The computer 615 regulates the actuator speed based on a volume of solution 707 in the vessel 702, inputted fluid delivery time and pressure, and feedback from various sensors that measure injection parameters. The delivery mechanism 627 can use an input/output device of the computer 615 to communicate status to the operator. The delivery mechanism 627 optionally includes various controls, such as start, stop, cancel, pause, and programming inputs. The programming inputs may alternatively be provided by the computer 615.

An occlusion pump 720 is in fluidic communication with an optional occluding balloon by way of a lumen coupled to an occluding coupling 722. The occluding pump 720 may be operated by the computer 615. The computer 615 may regulate the rate of inflation and the occlusion balloon pressure, based in part on the type of fluid used. Similarly, a stabilizer pump 730 may be in fluidic communication with a stabilizer on the catheter 609 through a stabilizer coupling 732. The stabilizer pump 730 may also be operated according by the computer 615. The occlusion and stabilizer pumps may be further regulated by the computer 615 based on feedback from various sensors that measure inflation parameters, such as fluid pressure, blood flow, pulse rate and the like. The pressure sensor is coupled to the delivery mechanism 627 by a connector 740 to provide tip pressure inputs to the algorithm.

The delivery mechanism 627 contains a solution 707 that includes a targetable nuclease. Any suitable nuclease may be in the solution 707 such as, for example, a transcription-activator like effector nuclease (TALEN), a zinc finger nuclease (ZFN), a meganuclease, or a CRISPR-associated nuclease (e.g., Cas5, Cas9, Cfp1, or a modified variant of Cas5, Cas9, Cfp1, etc.).

FIG. 8 shows a nuclease 801. Here, the nuclease 801 is illustrated as a ribonucleoprotein (RNP) that includes a Cas9/gRNA complex. The Cas9/gRNA complex includes a Cas9 endonuclease 825 in a complex with a guide RNA 805 (here shown as a single guide RNA sometimes dubbed an sgRNA), bound to the target 821 oncoviral nucleic acid via the guide sequence 809 of the guide RNA. The target 821 included to aid in understanding. Compositions of the invention according to some embodiments include the RNP (which provides the nuclease 801). In other embodiments, the nuclease may be delivered in the form of a protein or a nucleic acid (e.g., as mRNA or encoded on a plasmid).

In an aspect of the invention, the Cas9 endonuclease causes a double strand break in at least two locations in oncoviral nucleic acid. These two double strand breaks cause a fragment to be deleted. Even if viral repair pathways anneal the two ends, there will still be a deletion in the genome. One or more deletions using the mechanism will incapacitate the virus. The result is that the host cell will be free of viral infection.

In embodiments of the invention, nucleases cleave the genome of the target virus. A nuclease is an enzyme capable of cleaving the phosphodiester bonds between the nucleotide subunits of nucleic acids. Endonucleases are enzymes that cleave the phosphodiester bond within a polynucleotide chain. Some, such as deoxy-ribonuclease I, cut DNA relatively nonspecifically (without regard to sequence), while many, typically called restriction endonucleases or restriction enzymes, cleave only at very specific nucleotide sequences. In a preferred embodiment of the invention, the Cas9 nuclease is incorporated into the compositions and methods of the invention, however, it should be appreciated that any nuclease may be used.

The solution 707 may be delivered through the catheter 609.

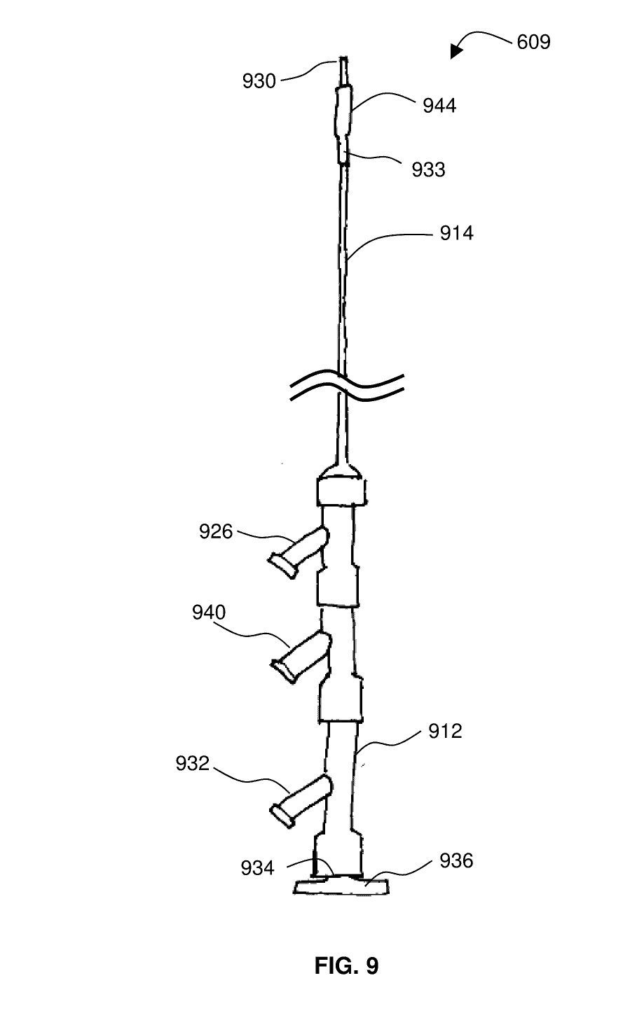

FIG. 9 shows catheter 609. The catheter 609 includes a proximal hub 912, which may be made of a rigid polycarbonate material. At the distal end of hub 912 is attached catheter tubing 914 which may be made of a material such as nylon, PEEK, or polytetraflouroethelyne. A distal portion of the catheter tube 914 includes one or a pair of catheter balloons 944. Inflation ports in the wall of catheter body 14 provide communication between the interior of the balloon 944 and a catheter lumen. The catheter 609 preferably includes an inflation for lumen for each balloon, a guidewire lumen, and a delivery lumen. An inflation port 926 provides temporary sealing communication with a syringe or any other desired source of compressed fluid in a manner conventional for balloon catheters.

A delivery lumen is provided within catheter body 914. The delivery lumen extends through the catheter to one or more apertures on a distal portion of the tubing 914. The delivery lumen communicates with an injection port 932, which may be coupled to the injection coupling 710. Additionally, the delivery lumen may communicates axially completely through hub 912 to extend through the proximal end 934 of the catheter as well. A conventional hemostasis screw 936 may be threadedly attached to the proximal end 934 of the catheter, to close off and seal the proximal end of the delivery lumen. Thus, a guide wire may be inserted through the entire length of the delivery lumen, to assist in catheter insertion in conventional manner. Then, after the catheter has been inserted to the desired position, the guide wire may be removed, and the hemostasis screw 936 applied. At this point, X-ray contrast or other desired solution may be administered through port 932, from where it is conveyed through the catheter, e.g., out of distal end 930.

Optionally, a second inflation lumen is provided in the catheter body 914 and accessible through the second inflation port 940. A second balloon may be carried by catheter body 914 and in communication with the second inflation lumen. Either balloon may be made, for example, of poly(ethylene terephthalate) or nylon.

The solution 707 may be delivered via catheter 609. Pressure may be applied as necessary causing the solution to migrate outwardly from the blood vessel. See Kamimura et al., 2015, Image-guided hydrodynamic gene delivery: current status and future directions, Pharmaceutics 7:213-223, incorporated by reference.

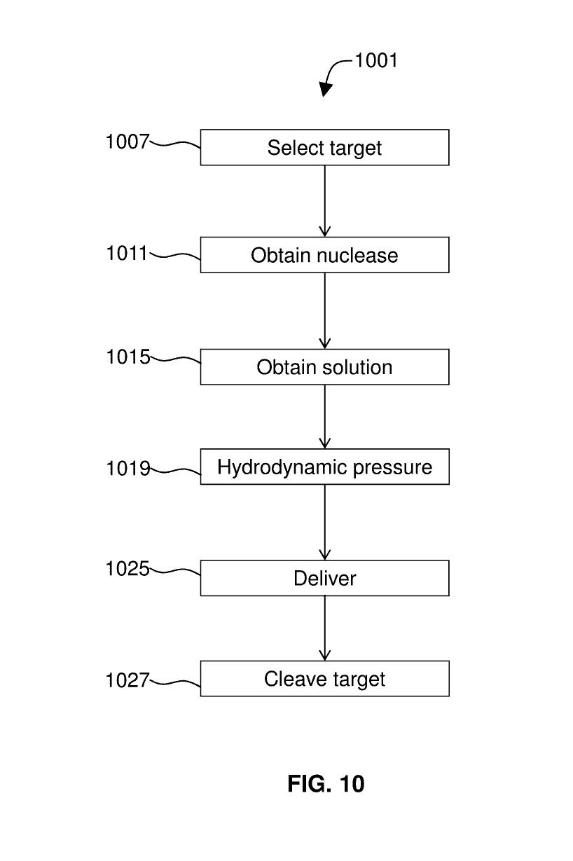

FIG. 10 diagrams a method 1001 of cleaving nucleic acid in a subject. The method 1001 may include selecting 1007 the target genetic material. In preferred embodiments, the target genetic material includes a portion of a viral genome from a virus such as a human papilloma virus (HPV), an Epstein-Barr virus (EBV), Kaposi's sarcoma-associated herpesvirus (KSHV), hepatitis B virus (HBV), hepatitis C virus (HCV), human T-cell lymphotrophic virus type I (HTLV-I), or Merkel cell polyomavirus (MCV).

The method 1001 includes obtaining 1011 a targetable nuclease such as a TALEN, a ZFN, a meganuclease, or a CRISPR-associated nuclease. In some embodiments, the targetable nuclease is a CRISPR-associated nuclease such as Cas9, Cfp1, or a modified variant thereof, where modified variant means having at least one amino acid substitution or indel but retaining at least 75% sequence identity with a wild type version thereof. The method 1001 includes obtaining 1015 a solution that include the targetable nuclease (e.g., in an enzymatically active form or optionally within a nanoparticle such as a liposome or cationic polymer). In certain embodiments, the solution includes the targetable nuclease as a CRISPR-associated nuclease in the form of a ribonucleoprotein that includes a guide RNA.

The method 1001 includes using a delivery system to generate 1019 hydrodynamic pressure and deliver 1025 the solution (that includes the targetable nuclease) to a blood vessel of the subject at a pressure sufficient to cause the targetable nuclease to enter cells proximal to the blood vessel. The method 1001 thus includes causing the targetable nuclease to cleave 1027 the target genetic material in a sequence-specific manner. Preferably, for the method 1001, systems of the invention are used to deliver the solution via an intravascular delivery catheter (e.g., by navigating the catheter through a femoral vein of the subject and into a hepatic vein or through a jugular vein of the subject and into a hepatic artery). Preferably, the catheter comprises a balloon catheter and delivery comprises navigating the balloon catheter to the blood vessel at a target location in the subject, inflating the balloon, and delivering the solution via a lumen in the balloon catheter. Most preferably, the solution is delivered at an injection volume substantially below 10% by BW, e.g., substantially below 100 mL/kg body weight, e.g., at less than 50 mL/kg, preferably between 10 and 20 ml/kg. The method 1001 may include imaging the blood vessel by injecting a phase contrast medium and using a radiographic imaging system and optionally further measuring pressure within the blood vessel via a sensor inserted therein, and using a computer comprising a processor coupled to non-transitory memory to control an injection speed of the solution according to the measured pressure.

It will be appreciated from the foregoing that the invention includes the method 101 for removing target genetic material from a subject. The method 101 includes delivering a solution comprising a nucleic acid to a blood vessel of a subject at a pressure sufficient to cause the nucleic acid to enter cells proximal to the blood vessel, wherein the nucleic acid comprises a gene for an enzyme that cuts target genetic material (e.g., a viral genome). In some embodiments of method 101, the enzyme is Cas9 and the solution is delivered at a pressure sufficient to generate pores in the cells proximal to the blood vessel. Preferably, the nucleic acid is a plasmid or mRNA comprising a Cas9 gene and at least one gene for a guide RNA (gRNA), the gRNA preferably being complementary to a portion of the viral genome.

In certain embodiments of the method 101, the viral genome is a hepatitis B genome and the plasmid contains genes for one or more sgRNAs targeting locations in the hepatitis B genome. For example, the one or more gRNAs may target locations in the hepatitis B genome selected from PreS1, DR1, DR2, a reverse transcriptase (RT) domain of polymerase, an Hbx, and the core ORF. In some embodiments, the one or more gRNAs comprise one selected from the group consisting of sgHBV-Core and sgHBV-PreS1.

In the method 101, the solution may be delivered via an intravascular delivery catheter (e.g., a balloon catheter). The delivery may include navigating the catheter through a femoral vein of the subject and into a hepatic vein or the delivery may include navigating the catheter through a jugular vein of the subject and into a hepatic artery. More specifically, the method 101 may include navigating the balloon catheter to the blood vessel at a target location in the subject, inflating the balloon, and delivering the solution via a lumen in the balloon catheter. The method 101 may further include delivering a radiographic contrast agent and imaging the balloon catheter in the target location.

In preferred embodiments of the method 101, the target genetic material is genome of a virus, and the nucleic acid is a plasmid comprising a cas9 gene and at least one sgRNA targeting the genome of the virus. The plasmid may further include a viral origin of replication (i.e., so that the plasmid is replicated in infected cells where its presence is beneficial).

Additionally, the invention includes the system 601 for delivering a nuclease. The system 601 includes a solution comprising a nucleic acid encoding a targetable nuclease, wherein the targetable nuclease comprises an enzyme that cuts target genetic material (e.g., a portion of a viral genome) in a sequence-specific manner; a delivery catheter dimensioned for insertion into a blood vessel of a subject; and a delivery mechanism operable to deliver the solution through the catheter at a pressure sufficient to cause the nucleic acid to enter cells proximal to the blood vessel. The delivery catheter preferably includes a balloon. The delivery catheter may include a delivery lumen for delivering the solution and an inflation lumen for inflating the balloon. The targetable nuclease may be a transcription-activator like effector nuclease, a zinc finger nuclease, or preferably a CRISPR-associated nuclease such as Cas9. The nucleic acid may also encode a gRNA. The viral genome may be of a virus selected from the group consisting of a human papilloma virus (HPV), an Epstein-Barr virus (EBV), Kaposi's sarcoma-associated herpesvirus (KSHV), hepatitis B virus (HBV), hepatitis C virus (HCV), human T-cell lymphotrophic virus type I (HTLV-I), and Merkel cell polyomavirus (MCV).

In certain embodiments, the system 601 includes a pressure sensor operable to measure pressure in the blood vessel. The delivery mechanism may include a computer comprising a processor coupled to non-transitory memory to control injection of the solution according to the measured pressure. Preferably, the delivery mechanism is operable to deliver the solution at an injection volume of less than 100 mL/1 kg body weight of the subject. The system 601 may include a phase contrast medium that can be injected into the blood vessel and a radiographic imaging subsystem for imaging the blood vessel with the contrast medium therein.

Optionally, the system 601 may be operable to control injection to deliver the solution when the cells proximal to the blood vessel are tumor cells and the system is operable to cause the solution to be delivered into the tumor cells.

In preferred embodiments of the system 601, the CRISPR-associated nuclease is Cas9, the gRNA includes a 20 nucleotide portion at least 60% complementary to a target within the viral genome, and wherein the 20 nucleotide portion is not complementary to any location in a human genome. Optionally, the nucleic acid encoding the targetable nuclease is mRNA.

Incorporation by Reference

References and citations to other documents, such as patents, patent applications, patent publications, journals, books, papers, web contents, have been made throughout this disclosure. All such documents are hereby incorporated herein by reference in their entirety for all purposes.

Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

In one embodiment, methods of the invention use hydrodynamic gene delivery to target the hepatitis B virus (HBV). More than 40% of the human population has been infected with HBV, giving rise to 240 million chronic HBV carriers and ca. 620,000 HBV-associated deaths annually. Human Hepatitis B virus (HBV), which is the prototype member of the family Hepadnaviridae, is a 42 nm partially double stranded DNA virus, composed of a 27 nm nucleocapsid core (HBcAg), surrounded by an outer lipoprotein coat (also called envelope) containing the surface antigen (HBsAg). The virus includes an enveloped virion containing 3 to 3.3 kb of relaxed circular, partially duplex DNA and virion-associated DNA-dependent polymerases that can repair the gap in the virion DNA template and has reverse transcriptase activities. HBV is a circular, partially double-stranded DNA virus of approximately 3200 by with four overlapping ORFs encoding the polymerase (P), core (C), surface (S) and X proteins. In infection, viral nucleocapsids enter the cell and reach the nucleus, where the viral genome is delivered. In the nucleus, second-strand DNA synthesis is completed and the gaps in both strands are repaired to yield a covalently closed circular DNA molecule that serves as a template for transcription of four viral RNAs that are 3.5, 2.4, 2.1, and 0.7 kb long. These transcripts are polyadenylated and transported to the cytoplasm, where they are translated into the viral nucleocapsid and precore antigen (C, pre-C), polymerase (P), envelope L (large), M (medium), S (small)), and transcriptional transactivating proteins (X). The envelope proteins insert themselves as integral membrane proteins into the lipid membrane of the endoplasmic reticulum (ER). The 3.5 kb species, spanning the entire genome and termed pregenomic RNA (pgRNA), is packaged together with HBV polymerase and a protein kinase into core particles where it serves as a template for reverse transcription of negative-strand DNA. The RNA to DNA conversion takes place inside the particles.

Numbering of basepairs on the HBV genome is based on the cleavage site for the restriction enzyme EcoR1 or at homologous sites, if the EcoR1 site is absent. However, other methods of numbering are also used, based on the start codon of the core protein or on the first base of the RNA pregenome. Every base pair in the HBV genome is involved in encoding at least one of the HBV protein. However, the genome also contains genetic elements which regulate levels of transcription, determine the site of polyadenylation, and even mark a specific transcript for encapsidation into the nucleocapsid. The four ORFs lead to the transcription and translation of seven different HBV proteins through use of varying in-frame start codons. For example, the small hepatitis B surface protein is generated when a ribosome begins translation at the ATG at position 155 of the adw genome. The middle hepatitis B surface protein is generated when a ribosome begins at an upstream ATG at position 3211, resulting in the addition of 55 amino acids onto the 5′ end of the protein.

ORF P occupies the majority of the genome and encodes for the hepatitis B polymerase protein. ORF S encodes the three surface proteins. ORF C encodes both the hepatitis e and core protein. ORF X encodes the hepatitis B X protein. The HBV genome contains many important promoter and signal regions necessary for viral replication to occur. The four ORFs transcription are controlled by four promoter elements (preS1, preS2, core and X), and two enhancer elements (Enh I and Enh II). All HBV transcripts share a common adenylation signal located in the region spanning 1916-1921 in the genome. Resulting transcripts range from 3.5 nucleotides to 0.9 nucleotides in length. Due to the location of the core/pregenomic promoter, the polyadenylation site is differentially utilized. The polyadenylation site is a hexanucleotide sequence (TATAAA) as opposed to the canonical eukaryotic polyadenylation signal sequence (AATAAA). The TATAAA is known to work inefficiently (9), suitable for differential use by HBV.

There are four known genes encoded by the genome, called C, X, P, and S. The core protein is coded for by gene C (HBcAg), and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigen (HBsAg). The HBsAg gene is one long open reading frame but contains three in-frame start (ATG) codons that divide the gene into three sections, pre-S1, pre-S2, and S. Because of the multiple start codons, polypeptides of three different sizes called large, middle, and small (pre-S1+pre-S2+S, pre-S2+S, or S) are produced. The function of the protein coded for by gene X is not fully understood but it is associated with the development of liver cancer. It stimulates genes that promote cell growth and inactivates growth regulating molecules.

HBV replicates its genome by reverse transcription of an RNA intermediate. The RNA templates is first converted into single-stranded DNA species (minus-strand DNA), which is subsequently used as templates for plus-strand DNA synthesis. DNA synthesis in HBV use oligoribonucleotides as primers for plus-strand DNA synthesis, which predominantly initiate at internal locations on the single-stranded DNA. The the primer is generated via an RNase H cleavage that is a sequence independent measurement from the 5′ end of the RNA template. This 18 nt RNA primer is annealed to the 3′ end of the minus-strand DNA with the 3′ end of the primer located within the 12 nt direct repeat, DR1. The majority of plus-strand DNA synthesis initiates from the 12 nt direct repeat, DR2, located near the other end of the minus-strand DNA as a result of primer translocation. The site of plus-strand priming has consequences. In situ priming results in a duplex linear (DL) DNA genome, whereas priming from DR2 can lead to the synthesis of a relaxed circular (RC) DNA genome following completion of a second template switch termed circularization. It remains unclear why hepadnaviruses have this added complexity for priming plus-strand DNA synthesis, but the mechanism of primer translocation is a potential therapeutic target. As viral replication is necessary for maintenance of the hepadnavirus (including the human pathogen, hepatitis B virus) chronic carrier state, understanding replication and uncovering therapeutic targets is critical for limiting disease in carriers.

Guide RNA against PreS1 locates at the 5′ end of the coding sequence. Endonuclease digestion will introduce insertion/deletion, which leads to frame shift of PreS1 translation. HBV replicates its genome through the form of long RNA, with identical repeats DR1 and DR2 at both ends, and RNA encapsidation signal epsilon at the 5′ end. The reverse transcriptase domain (RT) of the polymerase gene converts the RNA into DNA. Hbx protein is a key regulator of viral replication, as well as host cell functions. Digestion guided by RNA against RT will introduce insertion/deletion, which leads to frame shift of RT translation. Guide RNAs sgHbx and sgCore can not only lead to frame shift in the coding of Hbx and HBV core protein, but also deletion the whole region containing DR2-DR1-Epsilon. The four sgRNA in combination can also lead to systemic destruction of HBV genome into small pieces.

FIG. 2 shows key parts in the HBV genome targeted by CRISPR guide RNAs.

FIG. 3 shows a gel resulting from an in vitro CRISPR assay against HBV. Lane 1, 3, 6: PCR amplicons of HBV genome flanking RT, Hbx-Core, and PreS1. Lane 2, 4, 5, and 7: PCR amplicons treated with sgHBV-RT, sgHBV-Hbx, sgHBV-Core, sgHBV-PreS1.

The materials of the invention are thus shown to fragment and HBV virus genome.

What is claimed is:

1. A system for delivering a nuclease, the system comprising:
a solution comprising a targetable nuclease, wherein the targetable nuclease comprises an enzyme that cuts target genetic material in a sequence-specific manner;
a delivery catheter dimensioned for insertion into a blood vessel of a subject; and
a delivery mechanism operable to deliver the solution through the catheter at a pressure sufficient to cause the targetable nuclease to enter cells proximal to the blood vessel.

2. The system of claim 1, wherein the target genetic material comprises a portion of a viral genome.

3. The system of claim 2, wherein the delivery catheter comprises a balloon.

4. The system of claim 3, wherein delivery catheter comprises a delivery lumen for delivering the solution and an inflation lumen for inflating the balloon.

5. The system of claim 4, wherein the targetable nuclease is selected from the group consisting of a transcription-activator like effector nuclease and a zinc finger nuclease.

6. The system of claim 4, wherein the targetable nuclease is a CRISPR-associated nuclease.

7. The system of claim 6, wherein the CRISPR-associated nuclease is delivered as part of a ribonucleoprotein (RNP) that includes a Cas9 protein combined with a guide RNA (gRNA).

8. The system of claim 7, wherein the viral genome is of a virus selected from the group consisting of a human papilloma virus (HPV), an Epstein-Barr virus (EBV), Kaposi's sarcoma-associated herpesvirus (KSHV), hepatitis B virus (HBV), hepatitis C virus (HCV), human T-cell lymphotrophic virus type I (HTLV-I), and Merkel cell polyomavirus (MCV).

9. The system of claim 8, further comprising a pressure sensor operable to measure pressure in the blood vessel.

10. The system of claim 9, wherein the delivery mechanism comprises a computer comprising a processor coupled to non-transitory memory to control injection of the solution according to the measured pressure.

11. The system of claim 10, wherein the system is operable to control injection to deliver the solution when the cells proximal to the blood vessel are tumor cells and the system is operable to cause the solution to be delivered into the tumor cells.

12. The system of claim 8, wherein the delivery mechanism is operable to deliver the solution at an injection volume of less than 100 mL/1 kg body weight of the subject.

13. The system of claim 8, further comprising a phase contrast medium that can be injected into the blood vessel and a radiographic imaging subsystem for imaging the blood vessel with the contrast medium therein.

14. The system of claim 8, wherein the CRISPR-associated nuclease is Cas9, the gRNA includes a 20 nucleotide portion at least 60% complementary to a target within the viral genome, and wherein the 20 nucleotide portion is not complementary to any location in a human genome.

15. The system of claim 1, wherein the targetable nuclease is in a nanoparticle within the solution.

16. A method of cleaving nucleic acid in a subject, the method comprising:
delivering a solution comprising a targetable nuclease to a blood vessel of the subject at a pressure sufficient to cause the targetable nuclease to enter cells proximal to the blood vessel, wherein the targetable nuclease comprises an enzyme that cuts target genetic material in a sequence-specific manner.

17. The method of claim 16, wherein the target genetic material comprises a portion of a viral genome

18. The method of claim 17, wherein the solution is delivered via an intravascular delivery catheter.

19. The method of claim 18, wherein delivery comprises navigating the catheter through a femoral vein of the subject and into a hepatic vein.

20. The method of claim 18, wherein the delivery comprises navigating the catheter through a jugular vein of the subject and into a hepatic artery.

21. The method of claim 18, wherein the catheter comprises a balloon catheter.

22. The method of claim 21, wherein delivery comprises navigating the balloon catheter to the blood vessel at a target location in the subject, inflating the balloon, and delivering the solution via a lumen in the balloon catheter.

23. The method of claim 22, further comprising delivering the solution at an injection volume of less than 100 mL/1 kg body weight of the subject.

24. The method of claim 23, further comprising imaging the blood vessel by injecting a phase contrast medium and using a radiographic imaging system.

25. The method of claim 23, further comprising measuring pressure within the blood vessel via a sensor inserted therein, and using a computer comprising a processor coupled to non-transitory memory to control an injection speed of the solution according to the measured pressure.

26. The method of claim 23, wherein the targetable nuclease is selected from the group consisting of a transcription-activator like effector nuclease and a zinc finger nuclease.

27. The method of claim 23, wherein the targetable nuclease is a CRISPR-associated nuclease.

28. The method of claim 27, wherein the CRISPR-associated nuclease is delivered as part of a ribonucleoprotein that includes Cas9 protein combined with a gRNA.

29. The method of claim 27, wherein the viral genome is of a virus selected from the group consisting of a human papilloma virus (HPV), an Epstein-Barr virus (EBV), Kaposi's sarcoma-associated herpesvirus (KSHV), hepatitis B virus (HBV), hepatitis C virus (HCV), human T-cell lymphotrophic virus type I (HTLV-I), and Merkel cell polyomavirus (MCV).

30. The method of claim 29, wherein the cells proximal to the blood vessel are tumor cells

31. A method for removing target genetic material from a subject, the method comprising:
delivering a solution comprising a nucleic acid to a blood vessel of a subject at a pressure sufficient to cause the nucleic acid to enter cells proximal to the blood vessel, wherein the nucleic acid comprises a gene for an enzyme that cuts target genetic material.

32. A system for delivering a nuclease, the system comprising:
a solution comprising a nucleic acid encoding a targetable nuclease, wherein the targetable nuclease comprises an enzyme that cuts target genetic material in a sequence-specific manner;
a delivery catheter dimensioned for insertion into a blood vessel of a subject; and
a delivery mechanism operable to deliver the solution through the catheter at a pressure sufficient to cause the nucleic acid to enter cells proximal to the blood vessel.

33. The system of claim 32, wherein nucleic acid encoding the targetable nuclease is mRNA.

34. The system of claim 33, further comprising: a pressure sensor operable to measure pressure in the blood vessel; and a computer comprising a processor coupled to non-transitory memory to control injection of the solution according to the measured pressure, wherein the computer and the delivery mechanism are operable to deliver the solution and restrict the injection volume to less than 100 mL/1 kg body weight of the subject.

35. The system of claim 34, wherein the CRISPR-associated nuclease is Cas9, the gRNA includes a 20 nucleotide portion at least 60% complementary to a target within a viral genome, and wherein the 20 nucleotide portion is not complementary to any location in a human genome.
